SEQUENCE LISTING (58 sequences)

SEQ ID NO: 1 - DNA, 7249 bp, Artificial Sequence
  synthetic construct; M13mp18 phage DNA
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 2 - DNA, 32 bp, Artificial Sequence
  synthetic construct; folding DNA strand
ctttgaaaagaactggctcattatttaataaa

SEQ ID NO: 3 - DNA, 32 bp, Artificial Sequence
  synthetic construct; folding DNA strand
acggctacttacttagccggaacgctgaccaa

SEQ ID NO: 4 - DNA, 32 bp, Artificial Sequence
  synthetic construct; folding DNA strand
gagaatagcttttgcgggatcgtcgggtagca

SEQ ID NO: 5 - DNA, 32 bp, Artificial Sequence
  synthetic construct; folding DNA strand
acgttagtaaatgaattttctgtaagcggagt

SEQ ID NO: 6 - DNA, 32 bp, Artificial Sequence
  synthetic construct; folding DNA strand
acccaaatcaagttttttggggtcaaagaacg

SEQ ID NO: 7 - DNA, 32 bp, Artificial Sequence
  synthetic construct; folding DNA strand
tggactcccttttcaccagtgagacctgtcgt

SEQ ID NO: 8 - DNA, 32 bp, Artificial Sequence
  synthetic construct; folding DNA strand
gccagctgcctgcaggtcgactctgcaaggcg

SEQ ID NO: 9 - DNA, 32 bp, Artificial Sequence
  synthetic construct; folding DNA strand
attaagttcgcatcgtaaccgtgcgagtaaca

SEQ ID NO: 10 - DNA, 32 bp, Artificial Sequence
  synthetic construct; folding DNA strand
acccgtcgtcatatgtaccccggtaaaggcta

SEQ ID NO: 11 - protein, 11 aa, Artificial Sequence
  synthetic construct; tumor antigen polypeptide
SIINFEKLRRG

SEQ ID NO: 12 - DNA, 37 bp, Artificial Sequence
  synthetic construct; DNA in tumor antigen complex
aagcgctgtgcaagcctacgatggacacggtaacgac

SEQ ID NO: 13 - DNA, 175 bp, Artificial Sequence
  synthetic construct; template for immunlogic adjuvant
taatacgactcactataggtaaactctttgcgcacatggaagacgccaaaaacataaagaaaggcccggcgccattctatccgctggaagatggaaccgctggagagcaactgcataaggctatgaagagatacgccctggttcctggaacaattgcttttacagatgcacatat

SEQ ID NO: 14 - DNA, 160 bp, Artificial Sequence
  synthetic construct; template for immunlogic adjuvant
ttaatacgactcactataggatatgtgcatctgtaaaagcaattgttccaggaaccagggcgtatctcttcatagccttatgcagttgctctccagcggttccatcttccagcggatagaatggcgccgggcctttctttatgtttttggcgtcttccat

SEQ ID NO: 15 - DNA, 83 bp, Artificial Sequence
  synthetic construct; CpG immunologic adjuvant
gttagtgttagtgttagtttgcaagctgttgggttaccaccttcattggaaaacgttcttcggggcgttcttaggtggtaacc

SEQ ID NO: 16 - DNA, 37 bp, Artificial Sequence
  synthetic construct; capture DNA strand
gtcattaccgtgtccatcgtaggcttgcacagcgctt

SEQ ID NO: 17 - DNA, 69 bp, Artificial Sequence
  synthetic construct; capture DNA strand
caaaaatcattgctccttttgataagtttcatgtcattaccgtgtccatcgtaggcttgcacagcgctt

SEQ ID NO: 18 - DNA, 69 bp, Artificial Sequence
  synthetic construct; capture DNA strand
aaagattcagggggtaatagtaaaccataaatgtcattaccgtgtccatcgtaggcttgcacagcgctt

SEQ ID NO: 19 - DNA, 69 bp, Artificial Sequence
  synthetic construct; capture DNA strand
ggtagctaggataaaaatttttagttaacatcgtcattaccgtgtccatcgtaggcttgcacagcgctt

SEQ ID NO: 20 - DNA, 69 bp, Artificial Sequence
  synthetic construct; capture DNA strand
tttgccagatcagttgagatttagtggtttaagtcattaccgtgtccatcgtaggcttgcacagcgctt

SEQ ID NO: 21 - DNA, 69 bp, Artificial Sequence
  synthetic construct; capture DNA strand
gcaaatatcgcgtctggccttcctggcctcaggtcattaccgtgtccatcgtaggcttgcacagcgctt

SEQ ID NO: 22 - DNA, 69 bp, Artificial Sequence
  synthetic construct; capture DNA strand
tatattttagctgataaattaatgttgtataagtcattaccgtgtccatcgtaggcttgcacagcgctt

SEQ ID NO: 23 - DNA, 69 bp, Artificial Sequence
  synthetic construct; capture DNA strand
cattcaacgcgagaggcttttgcatattataggtcattaccgtgtccatcgtaggcttgcacagcgctt

SEQ ID NO: 24 - DNA, 69 bp, Artificial Sequence
  synthetic construct; capture DNA strand
accgttctaaatgcaatgcctgagaggtggcagtcattaccgtgtccatcgtaggcttgcacagcgctt

SEQ ID NO: 25 - DNA, 16 bp, Artificial Sequence
  synthetic construct; capture DNA strand
cacgcgtttctcaaat

SEQ ID NO: 26 - DNA, 48 bp, Artificial Sequence
  synthetic construct; capture DNA strand
gtgcgcaaagagtttacaaaattaaagtacggtgtctggaagaggtca

SEQ ID NO: 27 - DNA, 48 bp, Artificial Sequence
  synthetic construct; capture DNA strand
gtgcgcaaagagtttatttttgcgcagaaaacgagaatgaatgtttag

SEQ ID NO: 28 - DNA, 48 bp, Artificial Sequence
  synthetic construct; capture DNA strand
gtgcgcaaagagtttacgattttagaggacagatgaacggcgcgacct

SEQ ID NO: 29 - DNA, 48 bp, Artificial Sequence
  synthetic construct; capture DNA strand
gtgcgcaaagagtttagctccatgagaggctttgaggactagggagtt

SEQ ID NO: 30 - DNA, 48 bp, Artificial Sequence
  synthetic construct; capture DNA strand
gtgcgcaaagagtttatccatatacatacaggcaaggcaactttattt

SEQ ID NO: 31 - DNA, 48 bp, Artificial Sequence
  synthetic construct; capture DNA strand
gtgcgcaaagagtttaccaggcgcttaatcattgtgaattacaggtag

SEQ ID NO: 32 - DNA, 48 bp, Artificial Sequence
  synthetic construct; capture DNA strand
gtgcgcaaagagtttacaataaatacagttgattcccaatttagagag

SEQ ID NO: 33 - DNA, 48 bp, Artificial Sequence
  synthetic construct; capture DNA strand
gtgcgcaaagagtttatacctttaaggtctttaccctgacaaagaagt

SEQ ID NO: 34 - DNA, 21 bp, Artificial Sequence
  synthetic construct; capture DNA strand
ccctaaccctaaccctaaccc

SEQ ID NO: 35 - DNA, 53 bp, Artificial Sequence
  synthetic construct; capture DNA strand
ccctaaccctaaccctaacccagtaatcttaaattgggcttgagagaatacca

SEQ ID NO: 36 - DNA, 53 bp, Artificial Sequence
  synthetic construct; capture DNA strand
ccctaaccctaaccctaacccacgagtagtgacaagaaccggatataccaagc

SEQ ID NO: 37 - DNA, 53 bp, Artificial Sequence
  synthetic construct; capture DNA strand
ccctaaccctaaccctaacccccaaatcacttgccctgacgagaacgccaaaa

SEQ ID NO: 38 - DNA, 53 bp, Artificial Sequence
  synthetic construct; capture DNA strand
ccctaaccctaaccctaacccaaacgaaatgacccccagcgattattcattac

SEQ ID NO: 39 - DNA, 53 bp, Artificial Sequence
  synthetic construct; capture DNA strand
ccctaaccctaaccctaacccttcgccattgccggaaaccaggcattaaatca

SEQ ID NO: 40 - DNA, 53 bp, Artificial Sequence
  synthetic construct; capture DNA strand
ccctaaccctaaccctaacccgctcattttcgcattaaatttttgagcttaga

SEQ ID NO: 41 - DNA, 53 bp, Artificial Sequence
  synthetic construct; capture DNA strand
ccctaaccctaaccctaacccagacagtcattcaaaagggtgagaagctatat

SEQ ID NO: 42 - DNA, 53 bp, Artificial Sequence
  synthetic construct; capture DNA strand
ccctaaccctaaccctaaccccataacccgaggcatagtaagagctttttaag

SEQ ID NO: 43 - DNA, 62 bp, Artificial Sequence
  synthetic construct; DNA switch
acgttagtaaatgaatttctcttctcgtttgctcttctctttggtattgtctaagagaagag

SEQ ID NO: 44 - DNA, 35 bp, Artificial Sequence
  synthetic construct; DNA switch
acccaaatcaagttttaccagacaataccaaagag

SEQ ID NO: 45 - DNA, 62 bp, Artificial Sequence
  synthetic construct; DNA switch
cgtaacgatctaaagtttctcttctcgtttgctcttctctttggtattgtctaagagaagag

SEQ ID NO: 46 - DNA, 35 bp, Artificial Sequence
  synthetic construct; DNA switch
gtaaagcactaaatcgaccagacaataccaaagag

SEQ ID NO: 47 - DNA, 62 bp, Artificial Sequence
  synthetic construct; DNA switch
tgtagcattccacagattctcttctcgtttgctcttctctttggtattgtctaagagaagag

SEQ ID NO: 48 - DNA, 35 bp, Artificial Sequence
  synthetic construct; DNA switch
ccccgatttagagcttaccagacaataccaaagag

SEQ ID NO: 49 - DNA, 62 bp, Artificial Sequence
  synthetic construct; DNA switch
tgagtttcgtcaccagttctcttctcgtttgctcttctctttggtattgtctaagagaagag

SEQ ID NO: 50 - DNA, 35 bp, Artificial Sequence
  synthetic construct; DNA switch
gaacgtggcgagaaagaccagacaataccaaagag

SEQ ID NO: 51 - DNA, 62 bp, Artificial Sequence
  synthetic construct; DNA switch
caagcccaataggaacttctcttctcgtttgctcttctctttggtattgtctaagagaagag

SEQ ID NO: 52 - DNA, 35 bp, Artificial Sequence
  synthetic construct; DNA switch
cggccttgctggtaataccagacaataccaaagag

SEQ ID NO: 53 - DNA, 62 bp, Artificial Sequence
  synthetic construct; DNA switch
ctcagagccaccacccttctcttctcgtttgctcttctctttggtattgtctaagagaagag

SEQ ID NO: 54 - DNA, 35 bp, Artificial Sequence
  synthetic construct; DNA switch
ccgccagccattgcaaaccagacaataccaaagag

SEQ ID NO: 55 - DNA, 62 bp, Artificial Sequence
  synthetic construct; DNA switch
ccctcagaaccgccacttctcttctcgtttgctcttctctttggtattgtctaagagaagag

SEQ ID NO: 56 - DNA, 35 bp, Artificial Sequence
  synthetic construct; DNA switch
ggaaatacctacatttaccagacaataccaaagag

SEQ ID NO: 57 - DNA, 62 bp, Artificial Sequence
  synthetic construct; DNA switch
tatcaccgtactcaggttctcttctcgtttgctcttctctttggtattgtctaagagaagag

SEQ ID NO: 58 - DNA, 35 bp, Artificial Sequence
  synthetic construct; DNA switch
gaaatggattatttacaccagacaataccaaagag

(19) United States
(12) Patent Application Publication
(10) Pub. No.: US 2023/0355766 A1
(43) Pub. Date: Nov. 9, 2023

(54) DNA NANOVACCINE, PREPARATION METHOD THEREFOR AND USE THEREOF

(71) Applicant: NATIONAL CENTER FOR NANOSCIENCE AND TECHNOLOGY, Beijing (CN)

(72) Inventors: Baoquan Ding, Beijing (CN); Shaoli Liu, Beijing (CN); Qiao Jiang, Beijing (CN)

(21) Appl. No.: 17/774,695

(22) PCT Filed: Sep. 4, 2020
(86) PCT No.: PCT/CN2020/113489

§ 371 Date: May 5, 2022

(30) Foreign Application Priority Data
Sep. 9, 2019 (CN) 201910849707.2

(51) Int. Cl. A61K 39/00; A61P 37/04

(52) U.S. Cl. CPC A61K 39/464499; A61P 37/04; A61K 2039/53; A61K 2039/55561; B82Y 5/00

(57) ABSTRACT

Provided are a DNA nanovaccine, a preparation method therefor and the use thereof. The DNA nanovaccine comprises a DNA nanostructure, a tumor antigen polypeptide-DNA complex and an immunologic adjuvant, and the immunologic adjuvant comprises a double-stranded RNA immunologic adjuvant and/or a CpG immunologic adjuvant. In the present invention, a nanostructure is constructed, wherein the nanostructure is assembled from a DNA template, a DNA chain for assisting in folding and a capture DNA chain. By hybridizing the capture DNA chain with a functional component, the precise positioning and assembling of a tumor antigen molecule and an immunologic adjuvant molecule on the surface of the DNA self-assembled nanostructure is realized; in addition, a controllable DNA molecule “switch” is designed on one side of the tubular DNA nanostructure, which switch can respond to the acid environment of an endosome after entering an antigen-presenting cell, and open the tubular structure responsively to release the tumor antigen and the immunologic adjuvant molecule. The nanostructure has a tumor antigen-specific immunostimulatory effect and is a tumor vaccine used for the immunotherapy and prevention of various types of malignant tumors.

### CROSS REFERENCES TO RELATED APPLICATIONS

This application claims the priority of the Chinese invention patent application No. CN201910849707.2 filed on Sep. 9, 2019, which is incorporated herein by reference in its entirety.

### TECHINICAL FIELD

The invention belongs to the field of nano-medicine, and relates to a DNA nanovaccine, preparation method therefore and the use thereof, in particular to a tubular DNA nanovaccine loaded with tumor antigen and immunologic adjuvant, its preparation method and the use in tumor immunotherapy.

### BACKGROUND OF THE INVENTION

At present, there are problems of poor prognosis and low five-year survival rate in tumor treatment. Improving the therapeutic effect of tumor is a scientific problem that needs to be solved urgently. The rapid development of tumor immunotherapy methods in recent years has brought breakthrough results for tumor treatment. Tumor vaccines, especially individualized vaccines for specific patients, have attracted more and more attention from researchers and are expected to become a new tumor therapy in the near future program. However, the effectiveness of tumor vaccines remains limited. For example, free antigens may be rapidly cleared before being internalized by dendritic cells (DCs); in the absence of immunologic adjuvant, tumor vaccines can easily induce immune tolerance, limiting their therapeutic effects. How to improve the responsiveness of the immune system, further improve the therapeutic effect of tumor vaccines and reduce the occurrence of side effects, is an urgent problem to be solved. Recent studies have demonstrated that drug delivery systems can enhance the efficacy of immunostimulatory therapy by modulating the biodistribution, localization, in vivo stability, and release kinetics of immunotherapeutic drugs. Therefore, the use of biological materials to design new nano tumor vaccines has important research significance and broad the use prospects.

CN102614527A discloses an antiacid nano oral deoxyribonucleic acid (DNA) anti-tumor vaccine with potential of hydrogen (pH) sensitive characteristic and preparation method, the DNA oral vaccine is nano particles formed by combining chitosan with alginic acid for surface finish and encoded tumor specific antigen Legumain protein DNA plasmids. The DNA oral vaccine is capable of being efficiently phagocytized by dendritic cells and macrophage in peyer's patches and expressing the encoded tumor antigen to activate immune damage of a host for tumor cells. The nano particles have small toxic and side effects and a strong antigen presentation role. However, there are problems such as short cycle time and easy removal.

DNA origami nanotechnology is a novel and unique DNA self-assembly technology, which has been widely used in bottom-up preparation of nanoscale two-dimensional and three-dimensional DNA nanostructures. DNA nanostructures constructed by DNA nanotechnology have the characteristics of controllable structure and easy modification, and have broad application prospects in drug delivery and reversal of drug resistance. Therefore, it is of great practical significance to develop efficient, low toxicity, targeted and controllable DNA nanostructures as drug delivery carriers. Compared with traditional antitumor drug delivery carriers, DNA nanostructures have significant advantages in drug targeting delivery and controllable release due to their controllable structure and easy modification. DNA nanostructures are formed by DNA assembly and have good biocompatibility; DNA is assembled according to the principle of base complementary pairing, which has a high degree of structure predictability; the internal functional modification can effectively load a variety of active drugs, including genetic drugs, small molecule chemical drugs, proteins or antibodies, etc.; the complex high-level structure constructed by DNA nanotechnology has good structural stability in cell lysate, and plays an excellent protective role for the internally loaded drugs; short strand DNA hybridization on the outer surface can target and modify functional groups at specific sites, which enhances the targeting of the drug-loading system; DNA nanostructures can also be controlled to open and close under specific conditions by modifying nanoparticles to control drug release.

CN103656662A discloses a method for using a polypeptide-mediated DNA nanostructure as an antitumor drug carrier. The method is characterized in that a polypeptide with certain functions is connected to a DNA nanostructure; the prepared product is the combination of the DNA nanostructure and the polypeptide. After biomolecules are loaded to the surface of the DNA nanostructure and when the DNA nanostructure interacts with a cell, the polypeptide with the certain functions can mediate the DNA nanostructure loaded with the biomolecules to enter the cell or specifically be combined with a receptor on one surface of the cell so as to achieve the purpose of using the DNA nanostructure as the antitumor drug carrier. The method has a potential application value in the aspects of developing and researching the antitumor drug carrier, and improving the loading efficiency of the antitumor drug carrier. However, the DNA nanostructure can only be used as a drug carrier and does not have the effect of tumor treatment.

CN109675049A discloses a pH-induced drug sustained-release deoxyribonucleic acid (DNA) nanostructure as well as a preparation method and application thereof. Long single-stranded DNA is synthesized by using a rolling circle amplification technology, and is complemented and hybridized with G and C-rich DNA single strand (loading strand) so as to obtain DNA molecular aggregates with alternating single and double strands; the DNA molecular aggregates can carry a large amount of Dox. When the pH of the system decreases, the rolling circle amplification product is folded to form a triple helix configuration, the double strands of the DNA molecule aggregates are melted, the G and C-rich DNA single strand is released, and the inserted Dox is also released, so that the drug release is completed. The reversible intercalation and release of the Dox can be achieved by adjusting the pH. The method utilizes the DNA nanostructure with good biocompatibility as a drug carrier to increase the drug loading amount, and has the advantages of being low in cost, simple to operate, high in sensitivity to pH response, rapid in response, and the like, however, there is a problem of poor targeting to tumors.

Therefore, it has great significance in the field of tumor immunotherapy to construct a new type of tumor vaccine to realize the targeted delivery and controllable release of the vaccine, and to improve the therapeutic effect while reducing the occurrence of side effects.

### SUMMARY OF THE INVENTION

Therefore, the invention aims to overcome the defects in the prior art and provides a DNA nanovaccine, preparation method therefor and use thereof, the DNA nanovaccine hybridizes tumor antigen polypeptide molecules, double-stranded RNA adjuvant and CpG adjuvant to the interior of the DNA nanostructure through precise site design to form a tubular three-dimensional structure, and set controllable DNA switches on the surface of the tubular DNA nanostructure to respond to the acidic environment of the endosome in antigen presenting cell, realizing the target of tumor antigen polypeptides and immunologic adjuvant to deliver and controllable release, a new type of tumor vaccine with addressable, safe and efficient, controllable release and high medical value has been developed.

In order to achieve the above purpose, the technical scheme of the present invention is as follows:

The first aspect of the present invention provides a DNA nanovaccine, and the DNA nanovaccine comprises a DNA nanostructure, a tumor antigen polypeptide-DNA complex and an immunologic adjuvant;
the immunologic adjuvant includes a double-stranded RNA immunologic adjuvant and/or a CpG immunologic adjuvant.

In the present invention, the DNA nanovaccine has antigen-presenting cell endosome responsiveness, which can effectively induce tumor-specific immune responses and effectively inhibit tumor growth.

In the present invention, the alkynyl-modified tumor antigen polypeptide is connected with the azide-modified DNA strand through a “click” reaction to form tumor antigen polypeptide-DNA complex; the double-stranded RNA immunologic adjuvant uses DNA as a template, and is transcribed in vitro to form a single-stranded RNA, and two single-stranded RNAs were mixed in a molar ratio of 1:1 and then annealed to obtain double-stranded RNA.

In the present invention, the tumor antigen polypeptide and two immunologic adjuvants are used to play a synergistic effect, and the two signal pathways of TLR3 and TLR9 are simultaneously activated by precisely controlling the relative positions of the tumor antigen polypeptide and two immunologic adjuvants, which is better than the structure containing only one immunologic adjuvant.

Preferably, the DNA nanostructure is assembled by DNA template strand, assisted folding DNA strand and capture DNA strand.

Preferably, the DNA template strand includes M13mp18 phage genomic DNA and/or λ phage genomic DNA, more preferably M13mp18 phage genomic DNA;

In the present invention, the circular DNA single strand of M13mp18 phage is used as the main strand, and the excess short strand DNA is used as the auxiliary strand, through the hybridization and complementation of the main strand and the programmable auxiliary strand at specific positions, a two-dimensional rectangular lamellar DNA nanostructure is formed by folding.

In the present invention, the genetically modified M13 phage genomic DNA or asymmetric PCR amplification product can also be used as a DNA template strand for constructing DNA nanostructure.

Preferably, the nucleotide sequence of the M13mp18 phage genomic DNA is as shown in SEQ ID NO: 1;

SEQ ID NO: 1:
AATGCTACTACTATTAGTAGAATTGATGCCACCTTTTCAGCTCGCGCCCCAAATGAAAATATAGCTAAACAGGTTATTGA
CCATTTGCGAAATGTATCTAATGGTCAAACTAAATCTACTCGTTCGCAGAATTGGGAATCAACTGTTATATGGAATGAAA
CTTCCAGACACCGTACTTTAGTTGCATATTTAAAACATGTTGAGCTACAGCATTATATTCAGCAATTAAGCTCTAAGCCA
TCCGCAAAAATGACCTCTTATCAAAAGGAGCAATTAAAGGTACTCTCTAATCCTGACCTGTTGGAGTTTGCTTCCGGTCT
GGTTCGCTTTGAAGCTCGAATTAAAACGCGATATTTGAAGTCTTTCGGGCTTCCTCTTAATCTTTTTGATGCAATCCGCT
TTGCTTCTGACTATAATAGTCAGGGTAAAGACCTGATTTTTGATTTATGGTCATTCTCGTTTTCTGAACTGTTTAAAGCA
TTTGAGGGGGATTCAATGAATATTTATGACGATTCCGCAGTATTGGACGCTATCCAGTCTAAACATTTTACTATTACCCC
CTCTGGCAAAACTTCTTTTGCAAAAGCCTCTCGCTATTTTGGTTTTTATCGTCGTCTGGTAAACGAGGGTTATGATAGTG
TTGCTCTTACTATGCCTCGTAATTCCTTTTGGCGTTATGTATCTGCATTAGTTGAATGTGGTATTCCTAAATCTCAACTG
ATGAATCTTTCTACCTGTAATAATGTTGTTCCGTTAGTTCGTTTTATTAACGTAGATTTTTCTTCCCAACGTCCTGACTG
GTATAATGAGCCAGTTCTTAAAATCGCATAAGGTAATTCACAATGATTAAAGTTGAAATTAAACCATCTCAAGCCCAATT
TACTACTCGTTCTGGTGTTTCTCGTCAGGGCAAGCCTTATTCACTGAATGAGCAGCTTTGTTACGTTGATTTGGGTAATG
AATATCCGGTTCTTGTCAAGATTACTCTTGATGAAGGTCAGCCAGCCTATGCGCCTGGTCTGTACACCGTTCATCTGTCC
TCTTTCAAAGTTGGTCAGTTCGGTTCCCTTATGATTGACCGTCTGCGCCTCGTTCCGGCTAAGTAACATGGAGCAGGTCG
CGGATTTCGACACAATTTATCAGGCGATGATACAAATCTCCGTTGTACTTTGTTTCGCGCTTGGTATAATCGCTGGGGGT
CAAAGATGAGTGTTTTAGTGTATTCTTTTGCCTCTTTCGTTTTAGGTTGGTGCCTTCGTAGTGGCATTACGTATTTTACC
CGTTTAATGGAAACTTCCTCATGAAAAAGTCTTTAGTCCTCAAAGCCTCTGTAGCCGTTGCTACCCTCGTTCCGATGCTG
TCTTTCGCTGCTGAGGGTGACGATCCCGCAAAAGCGGCCTTTAACTCCCTGCAAGCCTCAGCGACCGAATATATCGGTTA
TGCGTGGGCGATGGTTGTTGTCATTGTCGGCGCAACTATCGGTATCAAGCTGTTTAAGAAATTCACCTCGAAAGCAAGCT
GATAAACCGATACAATTAAAGGCTCCTTTTGGAGCCTTTTTTTTGGAGATTTTCAACGTGAAAAAATTATTATTCGCAAT
TCCTTTAGTTGTTCCTTTCTATTCTCACTCCGCTGAAACTGTTGAAAGTTGTTTAGCAAAATCCCATACAGAAAATTCAT
TTACTAACGTCTGGAAAGACGACAAAACTTTAGATCGTTACGCTAACTATGAGGGCTGTCTGTGGAATGCTACAGGCGTT
GTAGTTTGTACTGGTGACGAAACTCAGTGTTACGGTACATGGGTTCCTATTGGGCTTGCTATCCCTGAAAATGAGGGTGG
TGGCTCTGAGGGTGGCGGTTCTGAGGGTGGCGGTTCTGAGGGTGGCGGTACTAAACCTCCTGAGTACGGTGATACACCTA
TTCCGGGCTATACTTATATCAACCCTCTCGACGGCACTTATCCGCCTGGTACTGAGCAAAACCCCGCTAATCCTAATCCT
TCTCTTGAGGAGTCTCAGCCTCTTAATACTTTCATGTTTCAGAATAATAGGTTCCGAAATAGGCAGGGGGCATTAACTGT
TTATACGGGCACTGTTACTCAAGGCACTGACCCCGTTAAAACTTATTACCAGTACACTCCTGTATCATCAAAAGCCATGT
ATGACGCTTACTGGAACGGTAAATTCAGAGACTGCGCTTTCCATTCTGGCTTTAATGAGGATTTATTTGTTTGTGAATAT
CAAGGCCAATCGTCTGACCTGCCTCAACCTCCTGTCAATGCTGGCGGCGGCTCTGGTGGTGGTTCTGGTGGCGGCTCTGA
GGGTGGTGGCTCTGAGGGTGGCGGTTCTGAGGGTGGCGGCTCTGAGGGAGGCGGTTCCGGTGGTGGCTCTGGTTCCGGTG
ATTTTGATTATGAAAAGATGGCAAACGCTAATAAGGGGGCTATGACCGAAAATGCCGATGAAAACGCGCTACAGTCTGAC
GCTAAAGGCAAACTTGATTCTGTCGCTACTGATTACGGTGCTGCTATCGATGGTTTCATTGGTGACGTTTCCGGCCTTGC
TAATGGTAATGGTGCTACTGGTGATTTTGCTGGCTCTAATTCCCAAATGGCTCAAGTCGGTGACGGTGATAATTCACCTT
TAATGAATAATTTCCGTCAATATTTACCTTCCCTCCCTCAATCGGTTGAATGTCGCCCTTTTGTCTTTGGCGCTGGTAAA
CCATATGAATTTTCTATTGATTGTGACAAAATAAACTTATTCCGTGGTGTCTTTGCGTTTCTTTTATATGTTGCCACCTT
TATGTATGTATTTTCTACGTTTGCTAACATACTGCGTAATAAGGAGTCTTAATCATGCCAGTTCTTTTGGGTATTCCGTT
ATTATTGCGTTTCCTCGGTTTCCTTCTGGTAACTTTGTTCGGCTATCTGCTTACTTTTCTTAAAAAGGGCTTCGGTAAGA
TAGCTATTGCTATTTCATTGTTTCTTGCTCTTATTATTGGGCTTAACTCAATTCTTGTGGGTTATCTCTCTGATATTAGC
GCTCAATTACCCTCTGACTTTGTTCAGGGTGTTCAGTTAATTCTCCCGTCTAATGCGCTTCCCTGTTTTTATGTTATTCT
CTCTGTAAAGGCTGCTATTTTCATTTTTGACGTTAAACAAAAAATCGTTTCTTATTTGGATTGGGATAAATAATATGGCT
GTTTATTTTGTAACTGGCAAATTAGGCTCTGGAAAGACGCTCGTTAGCGTTGGTAAGATTCAGGATAAAATTGTAGCTGG
GTGCAAAATAGCAACTAATCTTGATTTAAGGCTTCAAAACCTCCCGCAAGTCGGGAGGTTCGCTAAAACGCCTCGCGTTC
TTAGAATACCGGATAAGCCTTCTATATCTGATTTGCTTGCTATTGGGCGCGGTAATGATTCCTACGATGAAAATAAAAAC
GGCTTGCTTGTTCTCGATGAGTGCGGTACTTGGTTTAATACCCGTTCTTGGAATGATAAGGAAAGACAGCCGATTATTGA
TTGGTTTCTACATGCTCGTAAATTAGGATGGGATATTATTTTTCTTGTTCAGGACTTATCTATTGTTGATAAACAGGCGC
GTTCTGCATTAGCTGAACATGTTGTTTATTGTCGTCGTCTGGACAGAATTACTTTACCTTTTGTCGGTACTTTATATTCT
CTTATTACTGGCTCGAAAATGCCTCTGCCTAAATTACATGTTGGCGTTGTTAAATATGGCGATTCTCAATTAAGCCCTAC
TGTTGAGCGTTGGCTTTATACTGGTAAGAATTTGTATAACGCATATGATACTAAACAGGCTTTTTCTAGTAATTATGATT
CCGGTGTTTATTCTTATTTAACGCCTTATTTATCACACGGTCGGTATTTCAAACCATTAAATTTAGGTCAGAAGATGAAA
TTAACTAAAATATATTTGAAAAAGTTTTCTCGCGTTCTTTGTCTTGCGATTGGATTTGCATCAGCATTTACATATAGTTA
TATAACCCAACCTAAGCCGGAGGTTAAAAAGGTAGTCTCTCAGACCTATGATTTTGATAAATTCACTATTGACTCTTCTC
AGCGTCTTAATCTAAGCTATCGCTATGTTTTCAAGGATTCTAAGGGAAAATTAATTAATAGCGACGATTTACAGAAGCAA
GGTTATTCACTCACATATATTGATTTATGTACTGTTTCCATTAAAAAAGGTAATTCAAATGAAATTGTTAAATGTAATTA
ATTTTGTTTTCTTGATGTTTGTTTCATCATCTTCTTTTGCTCAGGTAATTGAAATGAATAATTCGCCTCTGCGCGATTTT
GTAACTTGGTATTCAAAGCAATCAGGCGAATCCGTTATTGTTTCTCCCGATGTAAAAGGTACTGTTACTGTATATTCATC
TGACGTTAAACCTGAAAATCTACGCAATTTCTTTATTTCTGTTTTACGTGCAAATAATTTTGATATGGTAGGTTCTAACC
CTTCCATTATTCAGAAGTATAATCCAAACAATCAGGATTATATTGATGAATTGCCATCATCTGATAATCAGGAATATGAT
GATAATTCCGCTCCTTCTGGTGGTTTCTTTGTTCCGCAAAATGATAATGTTACTCAAACTTTTAAAATTAATAACGTTCG
GGCAAAGGATTTAATACGAGTTGTCGAATTGTTTGTAAAGTCTAATACTTCTAAATCCTCAAATGTATTATCTATTGACG
GCTCTAATCTATTAGTTGTTAGTGCTCCTAAAGATATTTTAGATAACCTTCCTCAATTCCTTTCAACTGTTGATTTGCCA
ACTGACCAGATATTGATTGAGGGTTTGATATTTGAGGTTCAGCAAGGTGATGCTTTAGATTTTTCATTTGCTGCTGGCTC
TCAGCGTGGCACTGTTGCAGGCGGTGTTAATACTGACCGCCTCACCTCTGTTTTATCTTCTGCTGGTGGTTCGTTCGGTA
TTTTTAATGGCGATGTTTTAGGGCTATCAGTTCGCGCATTAAAGACTAATAGCCATTCAAAAATATTGTCTGTGCCACGT
ATTCTTACGCTTTCAGGTCAGAAGGGTTCTATCTCTGTTGGCCAGAATGTCCCTTTTATTACTGGTCGTGTGACTGGTGA
ATCTGCCAATGTAAATAATCCATTTCAGACGATTGAGCGTCAAAATGTAGGTATTTCCATGAGCGTTTTTCCTGTTGCAA
TGGCTGGCGGTAATATTGTTCTGGATATTACCAGCAAGGCCGATAGTTTGAGTTCTTCTACTCAGGCAAGTGATGTTATT
ACTAATCAAAGAAGTATTGCTACAACGGTTAATTTGCGTGATGGACAGACTCTTTTACTCGGTGGCCTCACTGATTATAA
AAACACTTCTCAGGATTCTGGCGTACCGTTCCTGTCTAAAATCCCTTTAATCGGCCTCCTGTTTAGCTCCCGCTCTGATT
CTAACGAGGAAAGCACGTTATACGTGCTCGTCAAAGCAACCATAGTACGCGCCCTGTAGCGGCGCATTAAGCGCGGCGGG
TGTGGTGGTTACGCGCAGCGTGACCGCTACACTTGCCAGCGCCCTAGCGCCCGCTCCTTTCGCTTTCTTCCCTTCCTTTC
TCGCCACGTTCGCCGGCTTTCCCCGTCAAGCTCTAAATCGGGGGCTCCCTTTAGGGTTCCGATTTAGTGCTTTACGGCAC
CTCGACCCCAAAAAACTTGATTTGGGTGATGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACTCAACCCTATCTCGGGCTATTCTTTTG
ATTTATAAGGGATTTTGCCGATTTCGGAACCACCATCAAACAGGATTTTCGCCTGCTGGGGCAAACCAGCGTGGACCGCT
TGCTGCAACTCTCTCAGGGCCAGGCGGTGAAGGGCAATCAGCTGTTGCCCGTCTCACTGGTGAAAAGAAAAACCACCCTG
GCGCCCAATACGCAAACCGCCTCTCCCCGCGCGTTGGCCGATTCATTAATGCAGCTGGCACGACAGGTTTCCCGACTGGA
AAGCGGGCAGTGAGCGCAACGCAATTAATGTGAGTTAGCTCACTCATTAGGCACCCCAGGCTTTACACTTTATGCTTCCG
GCTCGTATGTTGTGTGGAATTGTGAGCGGATAACAATTTCACACAGGAAACAGCTATGACCATGATTACGAATTCGAGCT
CGGTACCCGGGGATCCTCTAGAGTCGACCTGCAGGCATGCAAGCTTGGCACTGGCCGTCGTTTTACAACGTCGTGACTGG
GAAAACCCTGGCGTTACCCAACTTAATCGCCTTGCAGCACATCCCCCTTTCGCCAGCTGGCGTAATAGCGAAGAGGCCCG
CACCGATCGCCCTTCCCAACAGTTGCGCAGCCTGAATGGCGAATGGCGCTTTGCCTGGTTTCCGGCACCAGAAGCGGTGC
CGGAAAGCTGGCTGGAGTGCGATCTTCCTGAGGCCGATACTGTCGTCGTCCCCTCAAACTGGCAGATGCACGGTTACGAT
GCGCCCATCTACACCAACGTGACCTATCCCATTACGGTCAATCCGCCGTTTGTTCCCACGGAGAATCCGACGGGTTGTTA
CTCGCTCACATTTAATGTTGATGAAAGCTGGCTACAGGAAGGCCAGACGCGAATTATTTTTGATGGCGTTCCTATTGGTT
AAAAAATGAGCTGATTTAACAAAAATTTAATGCGAATTTTAACAAAATATTAACGTTTACAATTTAAATATTTGCTTATA
CAATCTTCCTGTTTTTGGGGCTTTTCTGATTATCAACCGGGGTACATATGATTGACATGCTAGTTTTACGATTACCGTTC
ATCGATTCTCTTGTTTGCTCCAGACTCTCAGGCAATGACCTGATAGCCTTTGTAGATCTCTCAAAAATAGCTACCCTCTC
CGGCATTAATTTATCAGCTAGAACGGTTGAATATCATATTGATGGTGATTTGACTGTCTCCGGCCTTTCTCACCCTTTTG
AATCTTTACCTACACATTACTCAGGCATTGCATTTAAAATATATGAGGGTTCTAAAAATTTTTATCCTTGCGTTGAAATA
AAGGCTTCTCCCGCAAAAGTATTACAGGGTCATAATGTTTTTGGTACAACCGATTTAGCTTTATGCTCTGAGGCTTTATT
GCTTAATTTTGCTAATTCTTTGCCTTGCCTGTATGATTTATTGGATGTT.

In the present invention, the assisted folding DNA strand is designed according to the article “Folding DNA to create nanoscale shapes and patterns. Nature, 2006, 440, 297-302”, and those skilled in the art can select the assisted folding DNA strand as needed, and the design of the capture DNA strand can be increased, decreased or altered as needed, and the design can be carried out on the entire plane of the DNA nanostructure.

Illustratively, the assisted folding DNA strand is exemplified as follows:

SEQ ID NO: 2:
CTTTGAAAAGAACTGGCTCATTATTTAATAAA;
SEQ ID NO: 3:
ACGGCTACTTACTTAGCCGGAACGCTGACCAA;
SEQ ID NO: 4:
GAGAATAGCTTTTGCGGGATCGTCGGGTAGCA;
SEQ ID NO: 5:
ACGTTAGTAAATGAATTTTCTGTAAGCGGAGT;
SEQ ID NO: 6:
ACCCAAATCAAGTTTTTTGGGGTCAAAGAACG;
SEQ ID NO: 7:
TGGACTCCCTTTTCACCAGTGAGACCTGTCGT;
SEQ ID NO: 8:
GCCAGCTGCCTGCAGGTCGACTCTGCAAGGCG;
SEQ ID NO: 9:
ATTAAGTTCGCATCGTAACCGTGCGAGTAACA;
SEQ ID NO: 10:
ACCCGTCGTCATATGTACCCCGGTAAAGGCTA.

Preferably, the capture DNA strand includes capture DNA strand I, capture DNA strand II and capture DNA strand III;

Preferably, the capture DNA strand I is formed by adding a capture sequence I complementary to the DNA sequence of the tumor antigen polypeptide-DNA complex at the 5′ end of the assisted folding DNA strand, the nucleotide sequence of the capture sequence I is as shown in SEQ ID NO: 16-24;

SEQ ID NO: 16:
GTCATTACCGTGTCCATCGTAGGCTTGCACAGCGCTT;
SEQ ID NO: 17:
CAAAAATCATTGCTCCTTTTGATAAGTTTCATGTCATTACCGTGTCCAT
CGTAGGCTTGCACAGCGCTT;
SEQ ID NO: 18:
AAAGATTCAGGGGGTAATAGTAAACCATAAATGTCATTACCGTGTCCAT
CGTAGGCTTGCACAGCGCTT;
SEQ ID NO: 19:
GGTAGCTAGGATAAAAATTTTTAGTTAACATCGTCATTACCGTGTCCAT
CGTAGGCTTGCACAGCGCTT;
SEQ ID NO: 20:
TTTGCCAGATCAGTTGAGATTTAGTGGTTTAAGTCATTACCGTGTCCAT
CGTAGGCTTGCACAGCGCTT;
SEQ ID NO: 21:
GCAAATATCGCGTCTGGCCTTCCTGGCCTCAGGTCATTACCGTGTCCAT
CGTAGGCTTGCACAGCGCTT;
SEQ ID NO: 22:
TATATTTTAGCTGATAAATTAATGTTGTATAAGTCATTACCGTGTCCAT
CGTAGGCTTGCACAGCGCTT;
SEQ ID NO: 23:
CATTCAACGCGAGAGGCTTTTGCATATTATAGGTCATTACCGTGTCCAT
CGTAGGCTTGCACAGCGCTT;
SEQ ID NO: 24:
ACCGTTCTAAATGCAATGCCTGAGAGGTGGCAGTCATTACCGTGTCCAT
CGTAGGCTTGCACAGCGCTT.

Preferably, the capture DNA strand II is formed by adding a capture sequence II complementary to the cohesive end sequence of the double-stranded RNA immunologic adjuvant at the 5′ end of the assisted folding DNA strand, the nucleotide sequence of the capture sequence II is as shown in SEQ ID NO: 25-33;

SEQ ID NO: 25:
CACGCGTTTCTCAAAT;
SEQ ID NO: 26:
GTGCGCAAAGAGTTTACAAAATTAAAGTACGGTGTCTGGAAGAGGTCA;
SEQ ID NO: 27:
GTGCGCAAAGAGTTTATTTTTGCGCAGAAAACGAGAATGAATGTTTAG;
SEQ ID NO: 28:
GTGCGCAAAGAGTTTACGATTTTAGAGGACAGATGAACGGCGCGACCT;
SEQ ID NO: 29:
GTGCGCAAAGAGTTTAGCTCCATGAGAGGCTTTGAGGACTAGGGAGTT;
SEQ ID NO: 30:
GTGCGCAAAGAGTTTATCCATATACATACAGGCAAGGCAACTTTATTT;
SEQ ID NO: 31:
GTGCGCAAAGAGTTTACCAGGCGCTTAATCATTGTGAATTACAGGTAG;
SEQ ID NO: 32:
GTGCGCAAAGAGTTTACAATAAATACAGTTGATTCCCAATTTAGAGAG;
SEQ ID NO: 33:
GTGCGCAAAGAGTTTATACCTTTAAGGTCTTTACCCTGACAAAGAAGT.

and/or preferably, the capture DNA strand III is formed by adding a capture sequence III complementary to the 5′ end extension sequence of the CpG immunologic adjuvant at the 5′ end of the assisted folding DNA strand, the nucleotide sequence of the capture sequence III is as shown in SEQ ID NO: 34-42;

SEQ ID NO: 34:
CCCTAACCCTAACCCTAACCC;
SEQ ID NO: 35:
CCCTAACCCTAACCCTAACCCAGTAATCTTAAATTGGGCTTGAGAGAAT
ACCA;
SEQ ID NO: 36:
CCCTAACCCTAACCCTAACCCACGAGTAGTGACAAGAACCGGATATACC
AAGC;
SEQ ID NO: 37:
CCCTAACCCTAACCCTAACCCCCAAATCACTTGCCCTGACGAGAACGCC
AAAA;
SEQ ID NO: 38:
CCCTAACCCTAACCCTAACCCAAACGAAATGACCCCCAGCGATTATTCA
TTAC;
SEQ ID NO: 39:
CCCTAACCCTAACCCTAACCCTTCGCCATTGCCGGAAACCAGGCATTAA
ATCA;
SEQ ID NO: 40:
CCCTAACCCTAACCCTAACCCGCTCATTTTCGCATTAAATTTTTGAGCT
TAGA;
SEQ ID NO: 41:
CCCTAACCCTAACCCTAACCCAGACAGTCATTCAAAAGGGTGAGAAGCT
ATAT;
SEQ ID NO: 42:
CCCTAACCCTAACCCTAACCC-CATAACCCGAGGCATAGTAAGAGCTTT
TTAAG.

According to the above design, it is ensured that the surface of each DNA nanostructure has a capture site, and the capture DNA strand at the capture site is complementary to the extended sequence of the tumor antigen polypeptide, double-stranded RNA immunologic adjuvant and CpG immunologic adjuvant. Through DNA annealed hybridization, the tumor antigen polypeptide, double-stranded RNA immunologic adjuvant and CpG immunologic adjuvant are assembled to specific sites on the surface of DNA nanostructure in a certain proportion.

Preferably, the tumor antigen polypeptide-DNA complex, the double-stranded RNA immunologic adjuvant and the CpG immunologic adjuvant were bound to the DNA nanostructure by capture DNA strand.

In the present invention, using the principle of base complementary pairing, the tumor antigen polypeptide-DNA complex, the double-stranded RNA (dsRNA) immunologic adjuvant and the CpG immunologic adjuvant were connected to the surface of the self-assembled two-dimensional lamina DNA nanostructure through site design, precise control of the number and relative position of tumor antigen polypeptide and immunologic adjuvant on the surface of lamellar DNA nanostructure.

Preferably, the number of the tumor antigen polypeptide-DNA complex is 10-30, for example, it can be 10, 11, 12, 13, 14, 15, 16, 17, 18, 19, 20, 21, 22, 23, 24, 25, 26, 27, 28, 29 or 30, more preferably 15-20;

Preferably, the number of the double-stranded RNA immunologic adjuvant is 10-30, for example, it can be 10, 11, 12, 13, 14, 15, 16, 17, 18, 19, 20, 21, 22, 23, 24, 25, 26, 27, 28, 29 or 30, more preferably 15-20; and/or

Preferably, the number of the CpG immunologic adjuvant is 10-30, for example, it can be 10, 11, 12, 13, 14, 15, 16, 17, 18, 19, 20, 21, 22, 23, 24, 25, 26, 27, 28, 29 or 30, more preferably 15-20.

In the present invention, the tumor antigen polypeptides are any tumor antigen polypeptides known to those skilled in the art, or may be new tumor antigen polypeptide sequences obtained by screening individualized tumor patients.

Preferably, the amino acid sequence of the tumor antigen polypeptide is as shown in SEQ ID NO: 11;
the amino acid sequence shown in SEQ ID NO: 11 is: SIINFEKLRRG.

In the present invention, the sequence of DNA in the tumor antigen polypeptide-DNA complex is shown in SEQ ID NO: 12;
the nucleotide sequence shown in SEQ ID NO: 12 is:

AAGCGCTGTGCAAGCCTACGATGGACACGGTAACGAC.

Exemplarily, the sequence of the tumor antigen polypeptide-DNA complex is:

SIINFEKLRRG-AAGCGCTGTGCAAGCCTACGATGGACACGGTAACGAC.

In the present invention, the nucleotide sequence of the DNA template for in vitro transcription and synthesis of double-stranded RNA immunologic adjuvant is as shown in SEQ ID NOs: 13 to 14;

SEQ ID NO: 13:
5′-TAATACGACTCACTATAGGTAAACTCTTTGCGCACATGGAAGACGC
CAAAAACATAAAGAAAGGCCCGGCGCCATTCTATCCGCTGGAAGATGGA
ACCGCTGGAGAGCAACTGCATAAGGCTATGAAGAGATACGCCCTGGTTC
CTGGAACAATTGCTTTTACAGATGCACATAT-3′;
SEQ ID NO: 14:
5′-TTAATACGACTCACTATAGGATATGTGCATCTGTAAAAGCAATTGT
TCCAGGAACCAGGGCGTATCTCTTCATAGCCTTATGCAGTTGCTCTCCA
GCGGTTCCATCTTCCAGCGGATAGAATGGCGCCGGGCCTTTCTTTATGT
TTTTGGCGTCTTCCAT-3′
Preferably, the nucleotide sequence of the CpG
immunologic adjuvant is as shown in SEQ ID
NO: 15;
SEQ ID NO: 15:
5′-GTTAGTGTTAGTGTTAGTTTGCAAGCTGTTGGGTTACCACCTTCAT
TGGAAAACGTTCTTCGGGGCGTTCTTAGGTGGTAACC-3′.

Preferably, the shape of the DNA nanovaccine comprises a rectangular two-dimensional structure and/or a tubular three-dimensional structure.

Preferably, the length of the rectangular two-dimensional structure is 80-100 nm, for example, it can be 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm or 100 nm, more preferably 90-100 nm.

Preferably, the width of the rectangular two-dimensional structure is 50-70 nm, for example, it can be 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm or 70 nm, more preferably 50-60 nm.

Preferably, the bottom diameter of the tubular three-dimensional structure is 10-25 nm, for example, it can be 10 nm, 11 nm, 12 nm, 13 nm, 14 nm, 15 nm, 16 nm, 17 nm, 18 nm, 19 nm, 20 nm, 21 nm, 22 nm, 23 nm, 24 nm or 25 nm, more preferably 19-20 nm. and/or

Preferably, the height of the tubular three-dimensional structure is 80-100 nm, for example, it can be 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm or 100 nm, more preferably 90-100 nm.

Further preferably, the DNA nanovaccine with the tubular three-dimensional structure has DNA switches.

In the present invention, DNA switches with acid environment responsiveness was hybridized on the two long sides of the rectangular lamella DNA nanostructure, the DNA switches were formed by the hybridization of two single-stranded DNA. The 3′ ends of the two single-stranded DNA were complementation to form an acid-responsive double-stranded “lock”, and the two-dimensional rectangular DNA nanostructures were coiled and closed to form a three-dimensional tubular DNA nanovaccine. Finally, a tubular DNA nanovaccine loaded with tumor antigen polypeptide and immunologic adjuvant and with controllable DNA switches was prepared. the tubular DNA nanovaccine realizes the loading, transportation and controllable release of tumor antigen polypeptide and immunologic adjuvant in lymph nodes, and is a new type of tumor immune vaccine.

In the present invention, the DNA switches respond to the acidic environment of the endosome in the antigen-presenting cell, control the opening of the tubular structure, and expose the tumor antigen polypeptide and two immunologic adjuvants therein.

More further preferably, the number of the DNA switches is 5-10, for example, it can be 5, 6, 7, 8, 9 or 10, more preferably 8-10. and/or

More further preferably, the nucleotide sequence of the DNA switches is as shown in SEQ ID NO: 43-58;

SEQ ID NO: 43:
ACGTTAGTAAATGAATTTCTCTTCTCGTTTGCTCTTCTCTTTGGTATTG
TCTAAGAGAAGAG;
SEQ ID NO: 44:
ACCCAAATCAAGTTTTACCAGACAATACCAAAGAG;
SEQ ID NO: 45:
CGTAACGATCTAAAGTTTCTCTTCTCGTTTGCTCTTCTCTTTGGTATTG
TCTAAGAGAAGAG;
SEQ ID NO: 46:
GTAAAGCACTAAATCG-ACCAGACAATACCAAAGAG;
SEQ ID NO: 47:
TGTAGCATTCCACAGATTCTCTTCTCGTTTGCTCTTCTCTTTGGTATTG
TCTAAGAGAAGAG;
SEQ ID NO: 48:
CCCCGATTTAGAGCTTACCAGACAATACCAAAGAG;
SEQ ID NO: 49:
TGAGTTTCGTCACCAGTTCTCTTCTCGTTTGCTCTTCTCTTTGGTATTG
TCTAAGAGAAGAG;
SEQ ID NO: 50:
GAACGTGGCGAGAAAGACCAGACAATACCAAAGAG;
SEQ ID NO: 51:
CAAGCCCAATAGGAACTTCTCTTCTCGTTTGCTCTTCTCTTTGGTATTG
TCTAAGAGAAGAG;
SEQ ID NO: 52:
CGGCCTTGCTGGTAATACCAGACAATACCAAAGAG;
SEQ ID NO: 53:
CTCAGAGCCACCACCCTTCTCTTCTCGTTTGCTCTTCTCTTTGGTATTG
TCTAAGAGAAGAG;
SEQ ID NO: 54:
CCGCCAGCCATTGCAAACCAGACAATACCAAAGAG;
SEQ ID NO: 55:
CCCTCAGAACCGCCACTTCTCTTCTCGTTTGCTCTTCTCTTTGGTATTG
TCTAAGAGAAGAG;
SEQ ID NO: 56:
GGAAATACCTACATTTACCAGACAATACCAAAGAG;
SEQ ID NO: 57:
TATCACCGTACTCAGGTTCTCTTCTCGTTTGCTCTTCTCTTTGGTATTGT
CTAAGAGAAGAG;
SEQ ID NO: 58:
GAAATGGATTATTTACACCAGACAATACCAAAGAG.

The tubular DNA nanovaccine used for tumor immunotherapy of the present invention is controllable for the release of the loaded tumor antigen polypeptide and immunologic adjuvant, the tubular DNA nanovaccine responds to the acid environment of the endosome after being taken up by antigen-presenting cells, so that the three active ingredients are released in the endosome in a controlled manner.

The second aspect of the present invention provides the preparation method of the DNA nanovaccine according to the first aspect, and the method includes the following steps:
(1) the DNA template strand, the assisted folding DNA strand and the capture DNA strand are mixed in the buffer in proportion, and annealed to obtain a rectangular DNA nanostructure;
(2) the annealed product obtained in step (1) is purified by centrifugation, mixed with the tumor antigen polypeptide-DNA complex, the double-stranded RNA immunologic adjuvant and the CpG immunologic adjuvant in proportion, and then annealed;
(3) the annealed product obtained in step (2) is mixed with the DNA switches in proportion and then annealed;
(4) the annealed product obtained in step (3) is purified by centrifugation to obtain a tubular DNA nanovaccine.

Preferably, the annealed conditions in step (1) are: the starting temperature is 95° C.-65° C., the end point temperature is 25° C.-4° C., each 1° C. is a gradient, each gradient stays for 5-10 minutes, and the annealed time is maintained at 2 -24 h, preferably 7-9 h.

Preferably, the starting temperature is 95-65° C., for example, it can be 95° C., 93° C., 91° C., 90° C., 87° C., 85° C., 83° C., 81° C., 80° C., 77° C., 75° C., 73° C., 71° C. ° C., 69° C., 67° C. or 65° C.

Preferably, the end point temperature is 25-4° C., for example, it can be 25° C., 24° C., 23° C., 21° C., 20° C., 19° C., 17° C., 15° C., 13° C., 11° C., 7° C., 5° C. ° C. or 4° C.

Preferably, the temperature of the annealed process is 2-24 h, for example, it can be 2 h, 4 h, 6 h, 7 h, 8 h, 9 h, 10 h, 12 h, 14 h, 16 h, 18 h, 20 h, 22 h or 24 h.

Preferably, the molar ratio of the DNA template strand, the assisted folding DNA strand and the capture DNA strand in step (1) is 1:(5-20):(5-20), for example, it can be 1:5:5, 1:7:7, 1:10:10, 1:15:15 or 1:20:20, preferably 1:(5-10):(5-10);

Preferably, the buffer described in step (1) is 1×TAE/Mg²⁺;

Preferably, the annealed conditions in step (2) and step (3) are: the starting temperature is 45-37° C., the end point temperature is 25-16° C., each 1° C. is a gradient, each gradient stays for 3-8 minutes, and carries out 3-10 cycles;

Preferably, the starting temperature is 45° C. to 37° C., for example, it can be 45° C., 44° C., 43° C., 42° C., 41° C., 40° C., 39° C., 38° C. or 37° C.

Preferably, the end point temperature is 25-16° C., for example, it can be 25° C., 24° C., 23° C., 22° C., 21° C., 20° C., 19° C., 18° C., 17° C. or 16° C.

Preferably, the number of the cycles is 3 to 10, for example, it can be 3, 4, 5, 6, 7, 8, 9 or 10.

Preferably, the molar ratio of the annealed product, the tumor antigen polypeptide-DNA complex, the double-stranded RNA immunologic adjuvant and the CpG immunologic adjuvant described in step (2) is 1:(2-10):(2-10):(2-10);

Preferably, the molar ratio of the annealed product in step (3) to the DNA switches is 1:(1-2); and/or

Preferably, the steps of centrifugation in steps (2) and (4) are as follows: mix the obtained annealed product with 1×TAE/Mg²⁺ buffer and add a 100 kDa spin column for centrifugation.

In the present invention, the inventor optimizes the reaction conditions through a large number of complex experiments, explores the influence of annealed temperature and reaction time on the nanostructure, and synergizes the effects of each step and condition, and finally successfully prepares a tubular DNA nanovaccine composite structure with excellent performance.

As a preferred technical solution, the invention provides the preparation method of the DNA nanovaccine according to the first aspect, the method comprises the following steps:
(1) the DNA template strand, the assisted folding DNA strand and the capture DNA strand are mixed in 1×TAE/Mg²⁺ buffer with a molar ratio of 1:(5-20):(5-20) for annealing, and the annealing conditions are: from 95° C. to 65° C., each 1° C. is a gradient, the residence time of each gradient is 5 min; from 65° C. to 25° C., each 1° C. is a gradient, the residence time of each temperature gradient is 10 min, the whole annealing process is 7-9 h, to obtain a rectangular DNA nanostructure;
(2) the annealed product obtained in step (1) was mixed with 1×TAE/Mg²⁺ buffer and added to a 100 kDa spin column, centrifuged, and then mixed with the tumor antigen polypeptide-DNA complex, the double-stranded RNA immunologic adjuvant and the CpG immunologic adjuvant with a molar ratio of 1:(2-10):(2-10):(2-10) and annealed, the annealed conditions are: from 45° C. to 25° C., each 1° C. is a gradient, and each gradient stays for 3 to 5 minutes, carries out 6 cycles;
(3) mixed and annealed the annealed product obtained in step (2) with the DNA switches in a molar ratio of 1:(1-2), the annealed conditions are: from 45° C. to 25° C., each 1° C. is a gradient, each gradient stays for 3-5 min, and carries out 6 cycles;
(4) the annealed product obtained in step (3) was mixed with 1×TAE/Mg²⁺ buffer, added to a 100 kDa spin column, and centrifuged to obtain a tubular DNA nanovaccine.

The third aspect of the present invention provides a pharmaceutical composition, the pharmaceutical composition comprises the DNA nanovaccine according to the first aspect;
preferably, the pharmaceutical composition also includes any one or a combination of at least two pharmaceutically acceptable carriers, excipients or diluents.

The fourth aspect of the present invention provides a use of the DNA nanovaccine according to the first aspect and/or the pharmaceutical composition according to the second aspect in preparing the tumor immunotherapy vaccine.

According to the use of the fourth aspect, wherein the tumor immunotherapy vaccine is a broad-spectrum anti-tumor vaccine;
preferably, the tumor is selected from one or more of the following: melanoma, breast cancer, colon cancer.

In the present invention, the tumor is not limited to a single type of malignant tumor, and the anti-tumor immunotherapy effect of DNA nanovaccine and/or pharmaceutical composition has a broad spectrum, and can be used for the treatment and prevention of various malignant tumors, such as melanoma, breast cancer, colon cancer and other malignant tumors.

The fifth aspect of the present invention provides a method for immunotherapy of tumors, the method comprises: administering the DNA nanovaccine according to the first aspect to a subject in need, the DNA nanovaccine prepared by the preparation method according to the second aspect and/or the pharmaceutical composition according to the third aspect;
preferably, the tumor is selected from one or more of the following: melanoma, breast cancer, colon cancer.

The sixth aspect of the present invention provides a method for preventing tumors, the method comprises: administering the DNA nanovaccine according to the first aspect to a subject in need, the DNA nanovaccine prepared by the preparation method according to the second aspect and/or the pharmaceutical composition according to the third aspect;
preferably, the tumor is selected from one or more of the following: melanoma, breast cancer, colon cancer.

The seventh aspect of the present invention provides a medicine for immunotherapy of tumors, the medicine comprises the DNA nanovaccine according to the first aspect, the DNA nanovaccine prepared by the preparation method according to the second aspect and/or the pharmaceutical composition according to the third aspect;
preferably, the tumor is selected from one or more of the following: melanoma, breast cancer, colon cancer.

The eighth aspect of the present invention provides a medicine for preventing tumors, the medicine comprises the DNA nanovaccine according to the first aspect, the DNA nanovaccine prepared by the preparation method according to the second aspect and/or the pharmaceutical composition according to the third aspect;
preferably, the tumor is selected from one or more of the following: melanoma, breast cancer, colon cancer.

Compared with the prior art, the present invention has the following beneficial effects:
(1) The present invention utilizes DNA nanotechnology, uses the circular DNA single strand of M13mp18 phage as the main strand, and uses excess short-strand DNA as the auxiliary strand, through the hybridization and complementarity of the main strand and the programmable auxiliary strand at a specific position, folding to form predictable and controllable two-dimensional rectangular lamella DNA nanostructure;
(2) The present invention is based on the principle of base complementary pairing, using the capture DNA strand to connect the tumor antigen polypeptide-DNA complex, double-stranded RNA immunologic adjuvant and CpG immunologic adjuvant on the surface of self-assembled two-dimensional lamella DNA nanostructure;
(3) The present invention hybridizes a DNA switch with acid environment responsiveness on the two long sides of a rectangular lamella DNA nanostructure, coiled and closed lamella DNA nanostructure to form three-dimensional tubular DNA nanovaccine,the tubular DNA nanovaccine loaded with tumor antigen polypeptide and immunologic adjuvant and with controllable DNA switch was prepared;
(4) The tubular DNA nanovaccine of the present invention can respond to an acidic environment in target cells, realize controllable conformational changes, release tumor antigen molecule and immunologic adjuvant, and enhance the immune stimulation effect;
(5) The present invention precisely controls the number and relative positions of tumor antigen polypeptide and two immunologic adjuvants on the surface of the lamella DNA nanostructure, and by adjusting the ratio of the three, the effect of activating the TLR3 and TLR9 signaling pathways at the same time is achieved, and the synergistic effect is exerted.
(6) The DNA nanovaccine of the present invention is used as a nano-scale molecular machine for the loading of tumor antigen and immunologic adjuvant, and is effectively transported to the lymph nodes for controllable release, which is expected to provide a new nanovaccine for tumor immunotherapy dosage form.

### BRIEF DESCRIPTION OF THE DRAWINGS

Hereinafter, the embodiments of the present invention will be described in detail with reference to the drawings, in which:

FIG. 1 shows an atomic force microscope morphology observation diagram of the rectangular lamella DNA nanostructure of example 1.

FIG. 2 shows the atomic force microscope morphology observation diagram of the tubular DNA nanovaccine of example 2.

FIG. 3 shows the targeting effect of the tubular DNA nanovaccine of example 3 on inguinal lymph nodes after subcutaneous injection.

FIG. 4 shows the inhibitory effect of the tubular DNA nanovaccine of example 4 on the lung metastasis of melanoma cells.

FIG. 5 shows the inhibitory effect of the tubular DNA nanovaccine of example 5 on the growth of mouse melanoma.

FIG. 6 shows a transmission electron microscope image of the tubular DNA nanostructure of example 6.

FIG. 7 shows a transmission electron microscope image of the tubular DNA nanostructure of example 7.

### BEST MODE FOR CARRYING OUT THE INVENTION

In order to further illustrate the technical means adopted by the present invention and its effects, the present invention will be further described below with reference to the embodiments and accompanying drawings. It should be understood that the specific embodiments described herein are only used to explain the present invention, but not to limit the present invention.

If the specific technology or condition is not indicated in the embodiment, it shall be performed according to the technology or condition described in the literature in the art or according to the product specification. The reagents or instruments that do not indicate the manufacturer are all conventional products that are commercially available from regular channels.

### EXAMPLE 1

### Preparation of Rectangular Lamellar DNA Nanostructure

Mixed M13mp18 template strand, assisted folding DNA strand (“staple” strand) and capture DNA strand at final concentrations of 20 nM and 100 nM for the template strand and the assisted folding DNA strand respectively; the mixture was slowly annealed by a gradient PCR instrument. The annealed conditions were as follows: from 95° C. to 65° C., each 1° C. is a gradient, and each gradient stays for 5 min; from 65° C. to 25° C., each 1° C. is a gradient, each temperature gradient stays for 10 min; the whole annealed process was 8 h, and the rectangular lamellar DNA nanostructure was obtained.

After the annealed procedure, the rectangular DNA nanostructure samples were taken out and centrifuged with a 100 kDa spin column (MWCO) to remove excess staple strands and capture DNA strands. The centrifugation conditions were as follows: added 350 μL 1×TAE-Mg buffer to 100 μL sample, centrifuge at 4800 rpm/min for 3 min, the volume of the remaining solution in the spin column is about 100 μL, and repeat the centrifugation 4 times. The final collected samples were analyzed by 1% agarose gel electrophoresis and the morphology of the lamellar structure was observed under an atomic force microscope.

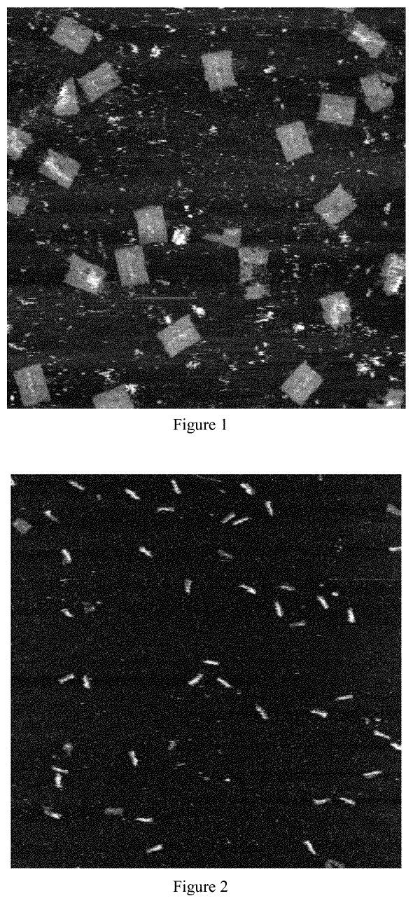

The results were shown in FIG. 1. the constructed DNA nanostructure has a rectangular lamellar structure, and the scanning results of atomic force microscopy showed that the rectangular DNA nanostructure is about 90-100 nm long and 60-80 nm wide, showing a regular rectangular structure.

### EXAMPLE 2

### Preparation of Tubular DNA Nanovaccine Loaded with Tumor Antigen and Immunologic Adjuvant

The purified rectangular lamellar DNA nanostructure solution, tumor antigen polypeptide-DNA complex, double-stranded RNA immunologic adjuvant, and CpG immunologic adjuvant were mixed uniformly according to the molar ratio of 1:5:5:5, and put into the gradient PCR instrument, slowly decreased from 45° C. to 25° C., each 1° C. is a gradient, and the residence time of each gradient was 5 min; 6 cycles were performed.

After the annealed procedure, the samples connected with the tumor antigen polypeptide and the two immunologic adjuvants were mixed with the DNA molecular “switch” in a molar ratio of 1:1 and annealed. The annealed conditions were as follows: from 45° C. to 25° C., each 1° C. is a gradient, and the residence time of each gradient was 5 min, and 6 cycles were carried out; and then the PCR products were separated by centrifugation with a 100 kDa spin column, purified and recovered by agarose gel electrophoresis, and the purified tubular DNA nanovaccine composite structure loaded with antigen and adjuvant was obtained.

The results were shown in FIG. 2, the morphology of the constructed tubular DNA nanostructure was characterized by atomic force microscopy (AFM).The structure is about 90-100 nm long and 20 nm wide, showing a regular tubular structure.

### EXAMPLE 3

### Evaluation of Lymph Node Targeting Effect of Tubular DNA Nanovaccine

A certain dose of the Cy5 fluorescently labeled tubular DNA nanovaccine of example 2 (the tumor antigen polypeptide is SEQ ID NO: 11: SIINFEKLRRG) was inoculated at the base of the tail of C57BL/6 mice, and the mice were anesthetized and killed 24 hours later, fluorescence imaging was performed on the inguinal lymph nodes of mice to evaluate the lymph node targeting effect of the tubular DNA nanovaccine.

The results were shown in FIG. 3, compared with the control group (fluorescently labeled DNA strands and fluorescently labeled rectangular DNA nanostructures), the tubular DNA nanostructure has a significant enrichment effect in the inguinal lymph nodes of mice, indicating that the tubular DNA nanostructure has obvious advantages as vaccine carriers.

### EXAMPLE 4

### Evaluation of Anti-Tumor Metastasis Effect of Tubular DNA Nanovaccine

2.0×10⁵ mouse B16-OVA melanoma cells were injected into C57BL/6 mice through the tail vein, and this time was counted as day 0; a certain dose of the tubular DNA nanovaccine of example 2 (tumor antigen polypeptide is SEQ ID NO: 11: SIINFEKLRRG) was inoculated into the tail base of the melanoma model mice on the 1st and 7th days, and the mice were killed on the 16th day, surgical removal of mouse lung tissue to observe the formation of metastasis in mouse lung tissue.

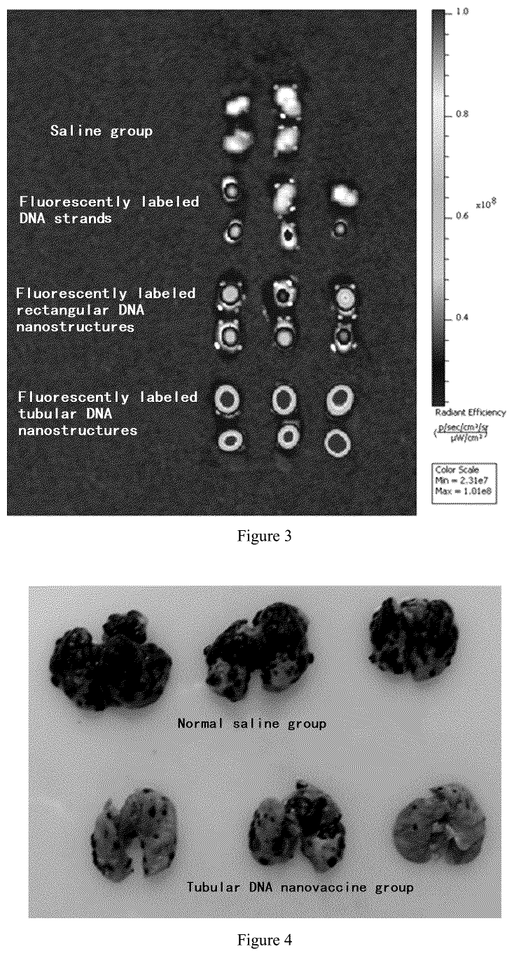

The results were shown in FIG. 4, compared with the control group (normal saline group), the number of metastases in the lung tissue of the experimental group (tubular DNA nanovaccine group) was significantly less, indicating that the tubular DNA nanovaccine has a significant inhibitory effect on tumor metastasis.

### EXAMPLE 5

### Evaluation of Antitumor Effect of Tubular DNA Nanovaccine

2.0×10⁵ mouse B16-OVA melanoma cells were inoculated on the back of C57BL/6 mice, and this time was counted as day 0; on the 4th day after inoculation, the melanoma was basically formed. On the 4th day and the 11th day, a certain dose of the tubular DNA nanovaccine of example 2 (the tumor antigen polypeptide is SEQ ID NO: 11: SIINFEKLRRG) was inoculated at the tail base of the mice respectively, the tumor volume was measured every 2 days, and the changes of tumor volume were statistically analyzed. The tumor volume was calculated according to the following formula, wherein d is the smallest diameter of the tumor, D is the largest diameter of the tumor, and the mice in the control group were injected with normal saline.

Volume=(d²×D)/2

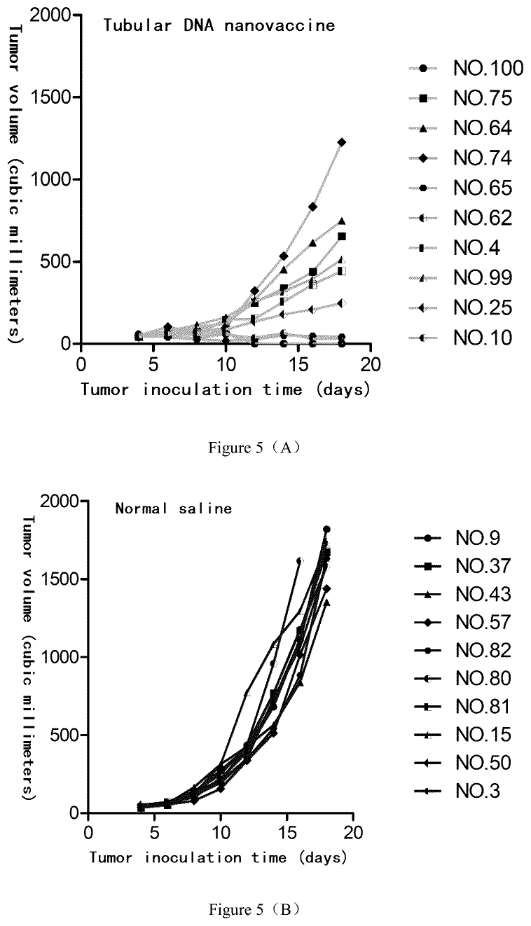

The results were shown in FIG. 5(A) and FIG. 5(B), compared with the normal saline group, the experimental group (tubular DNA nanovaccine group) can effectively inhibit the proliferation of melanoma in tumor-bearing mice, wherein the tumors of 4 mice completely regressed, showing a significant tumor treatment effect.

### EXAMPLE 6

### Preparation of Tubular DNA Nanovaccine Loaded with Tumor Antigen gp100 and Immunologic Adjuvant

For melanoma B16F10, the antigenic polypeptide gp10025-33 (KVPRNQDWL) was selected. Mixed the purified rectangular lamellar DNA nanostructure solution with tumor antigen polypeptide-DNA complex, double-stranded RNA immunologic adjuvant, and CpG immunologic adjuvant in a molar ratio of 1:5:5:5 uniformly, and put into the gradient PCR instrument, slowly decreased from 45° C. to 25° C., each 1° C. is a gradient, and the residence time of each gradient was 5 min; 6 cycles were performed.

After the annealed procedure, the samples connected with the tumor antigen polypeptide and the two immunologic adjuvants were mixed with the DNA molecular “switch” in a molar ratio of 1:1 and annealed. The annealed conditions were as follows: from 45° C. to 25° C., each 1° C. is a gradient, the residence time of each gradient was 5 min, and 6 cycles were performed; then the PCR products were separated by centrifugation with a 100 kDa spin column, purified and recovered by agarose gel electrophoresis, the purified tubular DNA nanovaccine composite structure loaded with antigen and adjuvant was obtained.

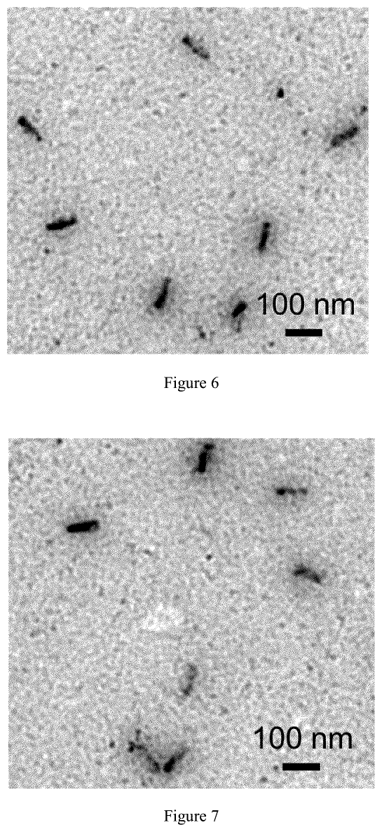

The results were shown in FIG. 6, the morphology of the constructed DNA nanostructure was characterized by transmission electron microscope, the structure was about 90-100 nm long and 20 nm wide, showing a regular tubular structure.

### EXAMPLE 7

### Preparation of Tubular DNA Nanovaccine Loaded with Tumor Antigen Adpgk and Immunologic Adjuvant

For colorectal tumors, the antigen Adpgk polypeptide (ASMTNMELM) was selected. Mixed the purified rectangular lamellar DNA nanostructure solution with tumor antigen polypeptide-DNA complex, double-stranded RNA immunologic adjuvant, and CpG immunologic adjuvant in a molar ratio of 1:5:5:5 uniformly, and put into the gradient PCR instrument, slowly decreased from 45° C. to 25° C., each 1° C. is a gradient, and the residence time of each gradient was 5 min; 6 cycles were performed.

After the annealed procedure, the samples connected with the tumor antigen polypeptide and the two immunologic adjuvants were mixed with the DNA molecular “switch” in a molar ratio of 1:1 and annealed. The annealed conditions were as follows: from 45° C. to 25° C., each 1° C. is a gradient, the residence time of each gradient was 5 min, and 6 cycles were performed; then the PCR products were separated by centrifugation with a 100 kDa spin column, purified and recovered by agarose gel electrophoresis, the purified tubular DNA nanovaccine composite structure loaded with antigen and adjuvant was obtained.

The results were shown in FIG. 7, the morphology of the constructed DNA nanostructure was characterized by transmission electron microscopy, the structure was about 90-100 nm long and 20 nm wide, showing a regular tubular structure.

### EXAMPLE 8

### Evaluation of Anti-Melanoma Effect of Tubular DNA Nanovaccine

2.0×10⁵ mouse B16-F10 melanoma cells were inoculated on the back of C57BL/6 mice, and this time was counted as day 0; on the 4th day after inoculation, the melanoma was basically formed; On the 4th day and the 11th day, a certain dose of the tubular DNA nanovaccine of example 6 was respectively inoculated at the tail base of the mice, the tumor volume was measured every 2 days, and the changes of tumor volume were statistically analyzed. The tumor volume was calculated according to the following formula, wherein d is the smallest diameter of the tumor, D is the largest diameter of the tumor, and the mice in the control group were injected with normal saline.

Volume=(d²×D)/2

Tumor dimensions were shown in Table 1 below. Compared with the control group, the DNA nanovaccine treatment group can effectively inhibit the proliferation of melanoma in tumor-bearing mice, showing a significant tumor therapeutic effect.

TABLE 1
Evaluation results of anti-melanoma
effect of tubular DNA nanovaccine

| Days after | Control group | DNA nanovaccine group |
|---|---|---|
| inoculation | tumor size (mm³) | tumor size (mm³) |
| 4 | 30.1 (±6.6) | 42.2 (±9.2) |
| 6 | 46.3 (±14.5) | 53.3 (±11.1) |
| 8 | 121.7 (±30.8) | 89.3 (±20.6) |
| 10 | 233.7 (±72.7) | 114.6 (±37.9) |
| 12 | 469.3 (±174.7) | 168.6 (±61.5) |
| 14 | 735.3 (±240.8) | 223.3 (±86.2) |
| 16 | 1125.2 (±337.6) | 363.5 (±144.4) |

### EXAMPLE 9

### Evaluation of Anti-Colorectal Tumor Effect of Tubular DNA Nanovaccine

1.0×10⁵ mouse MC-38 colorectal cancer cells were inoculated on the back of C57BL/6 mice, and this time was counted as day 0; on the 4th day after inoculation, the colorectal tumors were basically formed; On the 4th day and the 11th day, 100 nM (100 μL) of the tubular DNA nanovaccine of example 7 was inoculated at the tail base of the mice respectively, the tumor volume was measured every 2 days for 20 consecutive days, the changes of tumor volume were statistically analyzed. The tumor volume was calculated according to the following formula, wherein d is the smallest diameter of the tumor, D is the largest diameter of the tumor, and the mice in the control group were injected with normal saline.

Volume=(d²×D)/2

Tumor dimensions were shown in Table 2 below. Compared with the control group, the DNA nanovaccine treated group could effectively inhibit the proliferation of colorectal tumors in tumor-bearing mice, showing a significant tumor therapeutic effect.

TABLE 2
Evaluation results of the anti-colorectal
tumor effect of tubular DNA nanovaccine

| Days after | Control group | DNA nanovaccine group |
|---|---|---|
| inoculation | tumor size (mm³) | tumor size (mm³) |
| 4 | 68.5 (±10.8) | 62.6 (±14.7) |
| 6 | 102.2 (±27.0) | 83.3 (±47.1) |
| 8 | 170.0 (±28.0) | 112.0 (±20.6) |
| 10 | 256.4 (±49.1) | 118.0 (±50.3) |
| 12 | 317.3 (±65.8) | 139.1 (±76.7) |
| 14 | 406.7 (±95.8) | 154.6 (±85.7) |
| 16 | 502.9 (±112.3) | 177.6 (±98.3) |
| 18 | 591.0 (±144.1) | 198.7 (±118.6) |
| 20 | 760.2 (±187.8) | 213.9 (±113.1) |

In summary, the present invention uses the circular DNA single strand of the M13mp18 bacteriophage as the main strand, and the excess short-strand DNA as the auxiliary strand, through the hybridization and complementation of the main strand and the programmable auxiliary strand at a specific position, a two-dimensional rectangular lamellar DNA nanostructure is formed by folding. According to according to the principle of base complementary pairing, the tumor-specific antigen polypeptide, double-stranded RNA immunologic adjuvant and CpG immunologic adjuvant were connected to the surface of the self-assembled two-dimensional lamellar DNA nanostructure by using the capture DNA strand, then, acid-responsive DNA “switch” was hybridized on the two long sides of a rectangular lamellar DNA nanostructure, so that the rectangular structure was coiled and closed to form a tubular structure, a tubular DNA nanoparticle vaccine loaded with tumor antigen and immunologic adjuvant and controlled “switch” to respond to the acidic environment of antigen presenting cells in vivo was obtained. The nanoparticle vaccine has a bottom diameter of 19 nm and a height of 90 nm, which can be used as a nanoscale molecular machine for the loading of tumor antigen and immunologic adjuvant, and also can be effectively transported to lymph nodes for controllable release. It is expected to provide a new formulation of nanovaccine for tumor immunotherapy.

The applicant declares that the present invention illustrates the detailed method of the present invention through the above mentioned embodiments, but the present invention is not limited to the above mentioned detailed method, that is, it does not mean that the present invention must rely on the above mentioned detailed method to be implemented. Those skilled in the art should understand that any improvement of the present invention, the equivalent replacement of each raw material of the product of the present invention, the addition of auxiliary components, the selection of specific methods, etc., all fall within the protection scope and disclosure scope of the present invention.

1. A DNA nanovaccine comprising a DNA nanostructure, a tumor antigen polypeptide-DNA complex, and an immunologic adjuvant;
wherein the immunologic adjuvant comprises a double-stranded RNA immunologic adjuvant and/or a CpG immunologic adjuvant.

2. The DNA nanovaccine according to claim 1, wherein the DNA nanostructure is assembled by a DNA template strand, an assisted folding DNA strand, and a capture DNA strand.

3. The DNA nanovaccine according to claim 1, wherein the DNA template strand comprises M13mp18 phage genomic DNA and/or λ phage genomic DNA.

4. The DNA nanovaccine according to claim 1, wherein the capture DNA strand comprises a capture DNA strand I, a capture DNA strand II and a capture DNA strand III.

5. The DNA nanovaccine according to claim 1, wherein the tumor antigen polypeptide-DNA complex, the double-stranded RNA immunologic adjuvant, and the CpG immunologic adjuvant are bound to the DNA nanostructure by a capture DNA strand.

6. The DNA nanovaccine according to claim 1, wherein the shape of the DNA nanovaccine comprises a rectangular two-dimensional structure and/or a tubular three-dimensional structure.

7. A method for preparing the DNA nanovaccine according to claim 1 comprising the following steps:
(1) mixing the DNA template strand, the assisted folding DNA strand, and the capture DNA strand in a buffer in proportion, and annealing to obtain a rectangular DNA nanostructure;
(2) purifying the annealed product obtained in step (1) by centrifugation, mixing with the tumor antigen polypeptide-DNA complex, the double-stranded RNA immunologic adjuvants and the CpG immunologic adjuvants in proportion, and then annealing;
(3) mixing the annealed product obtained in step (2) with the DNA switches in proportion and then annealing; and
(4) purifying the annealed product obtained in step (3) by centrifugation to obtain a tubular DNA nanovaccine.

8. The method according to claim 7, wherein the method comprises the following steps:
(1) mixing the DNA template strand, the assisted folding DNA strands, and the capture DNA strand in a 1×TAE/Mg²⁺ buffer with a molar ratio of 1:(5-20):(5-20) for annealing, and the annealing conditions are: from 95° C. to 65° C., each 1° C. is a gradient, the residence time of each gradient is 5 min; from 65° C. to 25° C., each 1° C. is a gradient, the residence time of each temperature gradient is 10 min, the whole annealing process is 7-9 h, to obtain a rectangular DNA nanostructure;
(2) mixing the annealed product obtained in step (1) with a 1×TAE/Mg²⁺ buffer and adding to a 100 kDa spin column, centrifuging, and then mixing with the tumor antigen polypeptide-DNA complex, the double-stranded RNA immunologic adjuvants and the CpG immunologic adjuvants with a molar ratio of 1:(2-10):(2-10):(2-10) and annealed, the annealed conditions are: from 45° C. to 25° C., each 1° C. is a gradient, and each gradient stays for 3 to 5 minutes, carries out 6 cycles;
(3) mixing and annealing the annealed product obtained in step (2) with the DNA switches in a molar ratio of 1:(1-2), the annealing conditions are: from 45° C. to 25° C., each 1° C. is a gradient, each gradient stays for 3-5 min, and carries out 6 cycles;
(4) mixing the annealed product obtained in step (3) with 1×TAE/Mg²⁺ buffer, adding to a 100 kDa spin column, and centrifuging to obtain a tubular DNA nanovaccine.

9. A pharmaceutical composition comprising the DNA nanovaccine according to claim 1.

10. (canceled)

11. (canceled)

12. A method for treating a subject via immunotherapy of a tumor comprising administering the DNA nanovaccine according to claim 1 to a subject in need thereof, thereby treating a subject via immunotherapy of the tumor.

13. A method for preventing tumor growth in a subject comprising administering the DNA nanovaccine according to claim 1 to the subject in need thereof, thereby preventing tumor growth in the subject.

14. (canceled)

15. (canceled)

16. The DNA nanovaccine according to claim 3, wherein the nucleotide sequence of the M13mp18 phage genomic DNA is as shown in SEQ ID NO: 1.

17. The DNA nanovaccine according to claim 4, wherein, the capture DNA strand I is formed by adding a capture sequence I complementary to the DNA sequence of the tumor antigen polypeptide-DNA complex at the 5′ end of the assisted folding DNA strand, the nucleotide sequence of the capture sequence I is as shown in SEQ ID NO: 16-24;
the capture DNA strand II is formed by adding a capture sequence II complementary to the cohesive end sequence of the double-stranded RNA immunologic adjuvant at the 5′ end of the assisted folding DNA strand, the nucleotide sequence of the capture sequence II is as shown in SEQ ID NO: 25-33; and/or
the capture DNA strand III is formed by adding a capture sequence III complementary to the 5′ end extension sequence of the CpG immunologic adjuvant at the 5′ end of the assisted folding DNA strand, the nucleotide sequence of the capture sequence III is as shown in SEQ ID NO: 34-42.

18. The DNA nanovaccine according to claim 5, wherein,
the number of the tumor antigen polypeptide-DNA complex is 10-30;
the number of the double-stranded RNA immunologic adjuvant is 10-30;
the number of the CpG immunologic adjuvant is 10-30;
the amino acid sequence of the tumor antigen polypeptide is as shown in SEQ ID NO: 11;
the nucleotide sequence of the DNA template of the double-stranded RNA immunologic adjuvant is as shown in SEQ ID NO: 13-14; and/or
the nucleotide sequence of the CpG immunologic adjuvant is as shown in SEQ ID NO: 15.

19. The DNA nanovaccine according to claim 18, wherein,
the number of the tumor antigen polypeptide-DNA complex is 15-20;
the number of the double-stranded RNA immunologic adjuvant is 15-20; and/or
the number of the CpG immunologic adjuvant is 15-20.

20. The DNA nanovaccine according to claim 6, wherein,
the length of the rectangular two-dimensional structure is 80-100 nm;
the width of the rectangular two-dimensional structure is 50-70 nm;
the bottom diameter of the tubular three-dimensional structure is 10-25 nm; and/or
the height of the tubular three-dimensional structure is 80-100 nm.

21. The DNA nanovaccine according to claim 20, wherein,
the length of the rectangular two-dimensional structure is 90-100 nm;
the width of the rectangular two-dimensional structure is 50-60 nm;
the bottom diameter of the tubular three-dimensional structure is 19-20 nm; and/or
the height of the tubular three-dimensional structure is 90-100 nm.

22. The DNA nanovaccine according to claim 20, wherein the DNA nanovaccine with the tubular three-dimensional structure has DNA switches.

23. The DNA nanovaccine according to claim 22, wherein the number of the DNA switches is 5-10; and/or the nucleotide sequence of the DNA switches is as shown in SEQ ID NO: 43-58.

24. The method according to claim 12, wherein, the tumor is selected from one or more of the following: melanoma, breast cancer, colon cancer.
